(19) United States
(12) Patent Application Publication
(10) Pub. No.: US 2008/0004501 A1
(43) Pub. Date: Jan. 3, 2008

(54) Multirule Weight Control

(76) Inventor: Victor Gavrilov, Hackettstown, NJ (US)

Correspondence Address: Victor Gavrilov, 48 Countryside Apts, Hackettstown, NJ, 07840, US

(21) Appl. No.: 11/427,446

(22) Filed: Jun. 29, 2006

(51) Int. Cl. A61B 5/00

(52) U.S. Cl. 600/300

(57) ABSTRACT

A method for controlling a body weight and any weight-dependent parameters comprising consistent daily measurement of these parameters, setting monthly runs for monitoring, constructing a new control scale for each run based on individual variability of these parameters for the previous month, and identification of in-control, out-of-control and on-target states according to real nonrandom shifts of these parameters. The real shifts among their random fluctuations are detected by special control rules developed before for quality control. The method provides evaluation of progress and efficacy of the weight control over the run by measuring a mean value of results, variability of results and a score for weight shifts. The method may be embodied in paper template forms separated or inserted in any printed materials as well as in algorithms for computer programs inserted into a weight scale and other measuring devices, personal computer, PDA and other electronic devices, and in algorithms for programs provided direct data transfer from measuring devices to the computer.

### CROSS-REFERENCE TO RELATED APPLICATIONS

Not applicable.

### FEDERALLY SPONSORED RESEARCH OR DEVELOPMENT Not applicable.

### BACKGROUND OF THE INVENTION

1. Field of the Invention

This invention relates to a method for controlling a body weight and any weight-dependent parameters. More particularly, this invention relates to data analysis, provides the identification of in-control, out-of-control and on-target states and comprises a personalized control scale and special control rules. The early recognition of small nonrandom deviations of the weight and weight-dependent parameters among their random fluctuations significantly improves feedback control for any weight maintaining and especially weight loss programs due to promptly warning about a weight gain and supporting small progress in a weight loss. The method can be embodied on paper templates or in algorithms for computer programs.

2. Description of the Prior Art

Today America fails a battle with obesity: at least two in each three Americans desperately desire to get rid of extra pounds. The development of new weight control means and devices is the fast growing market. Despite different approaches weighing in keeps to be a unique reality check. However, the consistent weight monitoring does not play an essential role in almost all modern weight control programs which mainly focus on a diet and exercising.

The vital importance of weight monitoring becomes clear if a weight control system is described as to a modern theory of control. By this theory also named as the theory of automatic control all technical and natural controlled systems are based on the universal concept of a negative feedback loop and include five obligatory elements [R. M. MURRAY (editor) Control in an Information Rich World: Report of the Panel on Future Directions in Control, Dynamics and Systems, SIAM, 2002, pp. 7-13. http://www.cds.caltech.edu/%7Emurray/cdspanel/report/latest.pdf]:
(1) Object of regulation that described by a controlled variable or variables.
(2) Sensor, which needs to measure the controlled variable or an input signal and transfer its current value to analyzer.
(3) Analyzer, which provides analysis of input information, identifies deviations and makes a decision on correction actions.
(4) Effector represented by a process which produces an appropriate change in the controlled variable.
(5) Random influences (noise) causing unpredictable fluctuations of the controlled variable

### The appropriate specifications for elements of the weight control system are shown on FIG. 1 and in Table 1.

TABLE 1
Weight regulation as a negative feedback control system

| Element of negative | Element of weight control |
|---|---|
| feedback control system | system |
| Controlled variable | Body weight |
| Sensor | Weight scale |
| Effectors | 1/Eating |
| | 2/Physical activity |
| Source for random | Feelings: |
| deviations of controlled | 1/Emotional eating |
| variable | 2/Laziness |
| Analyzer | Mind: |
| | 1/Evaluation of Data |
| | 2/Decision Making |
| | 3/Effector Management |

Obviously, the body weight is the controlled variable and the weight scale serves as the sensor. There are two effectors with the opposite action on the body weight: eating, which is pushing weight up, and physical activity turning it down. The reason why no one can reach ideal balance between these two processes is human feelings which cause emotional eating and physical inactivity and lead to unpredictable weight fluctuations.

However, strictly speaking, both eating and physical activity are not real regulators. In turn, they are regulated by mind which represents the analyzer in a human body. Each analyzer works through special programs also called control rules which are essential like software for a computer. There are three types of control programs provided:

(1) Evaluation of data.

(2) Making a decision.

(3) Effector management.

Eventually, the efficacy of control rules determines the efficacy of the weight control. However, for now almost all efforts in the weight control are focused on the third program such as management of eating and physical activity. They include all diets, calorie balance control, diet supplements, exercise programs and exercise devises. On the contrary, almost nothing is known about control rules for weight evaluation due to common neglect of its role in the weight control.

Any variable has its own variability which determines a zone of random fluctuations. In practice, when a parameter is within that zone the analyzer evaluates it as “in control”. Only if the parameter goes beyond the fluctuation zone either above or below control limits the analyzer detects “out of control” event. If transfer this idea to the weight control directed to maintain or lose the weight three control zones should be set up as shown on FIG. 2:

(1) In Control, which is the zone of random weight fluctuations.

(2) Out of Control, which is the zone above the zone of random fluctuations.

(3) On Target, which is the zone below the zone of random fluctuations.

The designation of control zones is determined by a program for making a decision. If the purpose of weight correction is to gain the weight the On-Target zone will be above and Out-of-Control zone below In-Control zone. To keep it simple, the control rules just specify a control scale. Only then each weight value can be evaluated as in control, out of control or on target.

The traditional approach to the weight evaluation includes setting control scale for an initial weight but not for a purpose to analyze weight deviations. The standard method is based on the calculation of a body mass index (BMI) as a ratio between weight and square of height (BMI=W/H²). In 1998 the National Institute of Health proposed the well-known BMI control scale consisted of zones for underweight, desirable weight (normal range), overweight, obesity and severely obesity [BARBARA RAVAGE, KISS Guide to Weight Loss, DK ADULT, 2001, pp.24,25]. Due to population statistics each zone includes a significant weight interval which varies from 20 to 60 pound. This scale works well for a rough evaluation of the initial weight and setting a final goal if a person is out of a normal range. However, it does not work for monitoring small deviations because of the huge in-control zone. For example, a person with the initial weight 120 lb and height 173 cm can gain 55 lb and still stays within in-control zone with the final weight 175 lb.

The control scale for weight monitoring intends directly for evaluating a weight variation. It is known a body weight experiences up to 1-2 pound daily fluctuations. However, as a sole solution authors proposed just to exclude those fluctuations out of consideration at all. They recommended weighing once a week or even in longer intervals and then considering weight gain as real only if it exceeds 2.5 pound [JOY BAUER, Complete Idiot's Guide to Total Nutrition, Alpha; 4th edition, 2005, p.378] or 2% of initial weight [BARBARA RAVAGE, KISS Guide to Weight Loss, DK ADULT, 2001, pp.261,332-333]. The 2.5 lb in-control zone is much better compared to the 20-60 lb in-control zone for initial weight evaluation. However, even 2.5 lb detection limit for weight gain or loss does not provide effective weight monitoring.

The alternative approach to the weight control establishes a control scale for body fat as a proportion of the body weight (% F) using, for example, Bioelectrical Impedance Analysis. This scale designates underfat, healthy, overflat and obese zones [Live a Healthy Life, brochure, Tanita corp. 2004]. However, % F scale has the same flaws as the BMI scale: it intends only for initial % Fat evaluation and can not evaluate daily % Fat fluctuations due to a big % Fat interval for zones. Besides, there is no connection between %Fat and BMI scales.

The original method for the detection of nonrandom shifts among random fluctuations was developed in Quality Control (QC) for an analytical process DAMES O. WESTGARD, Basic QC Practices, Westgard QC Inc, 2th edition, 2002. (www.westgard.com) and U.S. Pat. No. 5,937,364 issued on Aug. 10, 1999 to Westgard at al.]. The QC aims to ensure the validity of analytical results by consistent testing control samples which have very high stability. Therefore the variability of the results is caused completely by variability in the measurement procedure. The QC sets up control rules to evaluate whether the result is within a zone of random fluctuation or in-control, or it is beyond that zone or out-of-control. Each out-of-control result is designated as an error of the measurement and suggests specific correction actions to be implemented.

The first part of QC refers to setting a control scale. It includes a data collection and statistics calculation such as Mean (M) and standard deviation (S) for a setting period of time. The key element of using those statistics in QC is an assumption that following results should have the same variability.

The second part of QC relates to using the control scale for data evaluation over a monitoring period of time. To evaluate the results a set of control rules so called as Multirule Quality Control or Westgard Rules was developed. Those rules were build on the idea that even a small deviation repeated several times reflects a true shift. Westgard just determined the relation between a degree of a deviation and a number of its consecutive repetitions to be detected as the nonrandom event. Each result or row of results violated any Control Rule is considered as nonrandom or an error in the procedure. For many years Multirule QC serves as a golden standard to control performance of all laboratory assays [MICHAEL L. BISHOP et al. Clinical Chemistry: principles, procedures, correlations. Lippincott Williams & Wilkins. 2005. pp.70-81].

It is important that there are no strong requirements for length of setting and especially monitoring periods. The only requirement for the setting period includes collecting at least 20 data which provides validity of statistical calculations. For stronger statistical validity of the control scale laboratories prefer to collect more data over 2, 3 and even 12 months and often calculate cumulative statistics when each new result is automatically added to a database for setting the control limits.

The idea of the present invention is to use a Multirule QC conception for a new area such as the weight control. However, the weight control and Quality Control have opposite variables to be analyzed. The QC analyses the variability of an analytical procedure by using constant object (control samples). The weight control should evaluate the variability of the object (body weight) by using constant procedure (weighing). Therefore the new requirements for setting control scale and monitoring results should be establish. Then the QC does not differentiate positive and negative deviations which is extremely important for the weight control. The QC detects only in-control and out-of-control states with the same rules for positive and negative shifts. The designation of control rules is complicated enough for a layperson where for example the rule for two consecutive results exceeded Mean+2S is designated as 2₂ₛ rule. The definitions of QC like a rejection rule and an error in the procedure can not be used for the weight control and new meanings for a violation of control rules should be defined. Finally, QC does not provide integral evaluation of results for the monitoring period of time focusing on the calculation of statistics only for a setting period.

### SUMMARY OF THE INVENTION

The subject of the invention is to provide the easy to use method of the weight control by identifying in-control, out-of-control and on-target states according to real shifts of the randomly variable weight and weight-dependent parameters. The method comprises consistent daily measurements of these parameters for the fasting body in the morning and their arrangement in monthly cycles. For each calendar month a special control scale is set up by using statistics such as mean and standard deviation calculated for the last results of the previous calendar month with no evident trend and shift. Each current result for the body weight or weight-dependent parameters within month analyzed is evaluated by using the control scale and a set of control rules developed before for Quality Control. A result or a row of consecutive results violated any of control rules are evaluated as a real or nonrandom shift where out-of-control and on target zone assignment depends on a goal of weight correction. Otherwise, the result is considered as in-control or a random fluctuation.

The embodiments of the invention comprise special template forms to track and evaluate results of measurements. The template chart includes color code for different zones of control scale to make visual detection of weight shifts easier. The method may be embodied in paper template forms separated or inserted in any printed materials as well as in algorithms for computer programs inserted into a weight scale and other measuring devices, personal computer, PDA and other electronic devices, and in algorithms for programs provided direct data transfer from measuring devices to the computer.

### OBJECTS AND ADVANTAGES.

Accordingly, several objects and advantages of the present invention are:
(1) to provide a new way for control of the body weight and weight-dependent parameters which comprises consistent daily measurements of these parameters for the fasting body, monthly runs with a new control scale for each month and a set of control rules to assess in-control, out-of control and on-target states;
(2) to provide new weight-dependent parameters to be controlled such as fat mass (FM) comprising % Fat multiplied by body weight(W) FM=% Fat*W and fat mass index (FMI) comprising fat mass divided by square of height FMI=FM/H² or FMI=% F*W/H² which combine BMI and % Fat evaluation together;
(3) to set up a new control scale with the real detection limit for a shift of these parameters which determined by the interval of random fluctuations. The control scale is constructed for each following month based on individual variability of these parameters for the previous month using a mean and standard deviation for the last results of that month with no evident trend or shift;
(4) to set up three control zones such as in-control, out-of-control and on-target instead of two in-control and out-of-control as to QC. Those zones better correspond to the goal of the weight control;
(5) to provide a high sensitive detection of the real shifts for these parameters among their random fluctuations by using special control rules developed before for Quality Control. It results in lowering detection limit for weight shifts as low as 0.2 lb instead of 2.5 lb as for prior art;
(6) to set up different groups of control rules specified for positive and negative shifts;
(7) to simplify designation of control rules and define new meaning for their violation.
(8) to provide easy to use template forms and charts to track and evaluate results with the color code for different zones of the control scale;
(9) to statistically evaluate progress in the weight control as a mean and variability of results for a whole run or for the last results of the month with no evident trend and shift. The decreasing variability should be considered as the first step to take the weight over control. On the contrary, increasing variability makes it worse by rising chances the weight slips out of control;
(10) to evaluate efficacy of the weight control by calculating total score for all weight shifts over the monthly run;
(11) to provide a simple algorithm to be used for computer programs inserted into the weight scale and other measuring devices, personal computer, PDA or other electronic devises.

Other objects and aspects of the present invention will become apparent from the following detailed description of the preferred embodiments, the appended claims and the accompanying drawings.

### DRAWING FIGURES

FIG. 1 shows a scheme of the weight control as a negative feedback controlled system.

FIG. 2 shows a scheme of control zones for the weight control directed to maintain or lose weight.

FIG. 3. shows setting a control scale.

FIG. 4 shows graphical presentation of control rules for a weight gain.

FIG. 5 shows graphical presentation of control rules for a weight loss.

FIG. 6 shows template forms for logging results on over monthly run.

FIG. 7 shows a template chart for plotting results on over monthly run.

FIG. 8 shows a template chart for tracking summary results for a month.

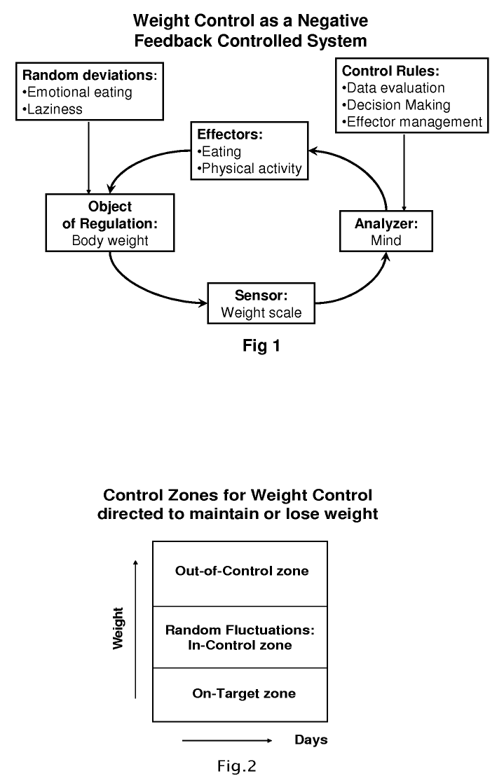

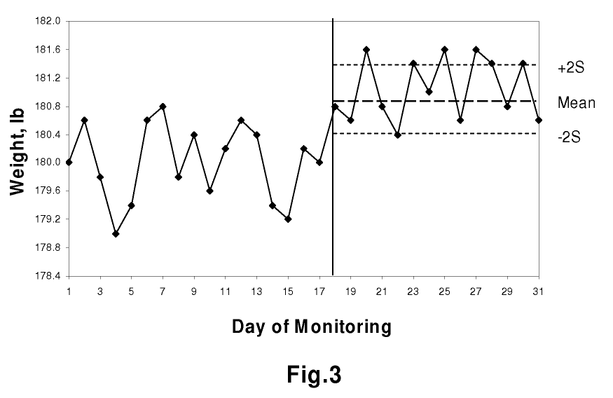

### DESCRIPTION OF THE INVENTION

The execution of the present invention comprises following procedures.

### 1. Choosing Control Parameters.

An individual chooses parameters for the weight control. By using a standard weight scale with an option for % Fat measurement everyone can directly measure the most popular parameters such as the body weight (W) and % Fat. The BMI is calculated from initial data for the weight. In addition to known BMI two new parameters are proposed to estimate:

a/Fat mass (FM) determined as % Fat multiplied by body weight: FM=% Fat*W b/Fat Mass Index (FMI) determined as fat mass divided by square of height: FMI=FM/H²=% Fat*W/H².

The new parameters FM and FMI integrate two known systems for weight evaluation such as BMI and % Fat together and conform with overweight assessment better than each of known indexes separately.

Besides, other weight-related parameters including now available for some weight scales can be used. They comprise but are not limited to %Body Water, Muscle Mass, Bone Mass and Visceral Fat.

The term “weight” using below in the text means the same for any other weight-dependent parameter.

### 2. Consistent Daily Measurement of the Weight for the Fasting Body.

The first rule for any statistical analysis is to collect sufficient number of data. It is a reason why a person should weigh in as much as possible. However, since the weight has fast in-day fluctuations depending on eating and daily activity there is no sense to make measurements within a day. The measurement of fasting morning value is the mandatory approach for an estimation of any metabolically active parameter such as glucose, lipoproteins, albumin and others. Weighing the fasting weight should also be a first priority due to its high dependence on metabolism. Then in-day fluctuations have minimal impact on results.

Of course, the person can skip a measurement at some days, but the less measurements one makes the less the precision for the following statistical calculation will be.

### 1. Setting up Monthly Run for Monitoring Period.

Despite deceptive simplicity of this question here is the key to the invention. The Quality Control has a deal with measurements of the constant object such as control samples. It does not matter whether setting and monitoring periods are one or three months or even twelve months: the statistical power only gets stronger for longer time.

On the contrary, the object of weight control can have very high variability and long trends up and down. The length of the monitoring period defines how long the control scale is valid. Using the old scale for a long time can become incorrect if a person starts consistently loosing or gaining weight. Very frequent setting a new scale is like continuous changing the rules during the game in your favor. In that case the weight likely will be in control zone all the time with a variation of that zone itself.

To sum it up, keeping constant length for monitoring period is mandatory for the present invention. This period is defined as a calendar month due to its easy usage. Each month a person starts a new run with a new control scale determined by progress in the previous month. This way is very useful to focus each individual on monthly progress while unwanted weight shifts can be detected and promptly corrected within a run. It also completely changes a strategy for weight control: the person gets psychological relief from being concentrated on each weight jump up at each day since he or she has time to correct it. On the other hands, the monitoring period is short enough to periodically evaluate the current progress and make correction decisions.

### 2. Setting up a Control Scale.

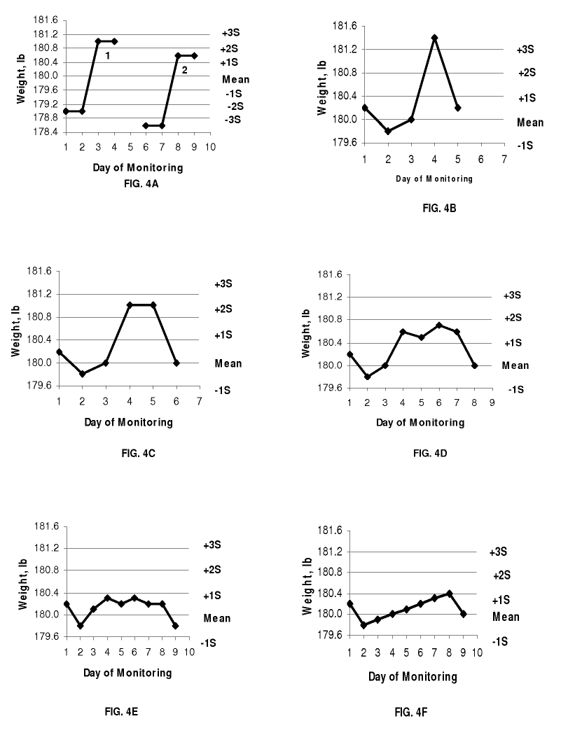

The person collects data for the first month of monitoring and plots them on the chart. An example is shown on FIG. 3. Here is the next principal difference of the invention from the QC method. As per QC all the data for a month should be used to set up the control scale with required statistical validity. For the example above it gets Mean=180.5 lb and a 95% confidence interval for random fluctuations 2S=1.0 lb.

However, this way does not take into account any significant weight trends which may happen to the end of the month. As for biological sense the last results are more important than the first ones. Therefore the setting period should include only last days of monitoring with no visual trends. The lower limit of 7 days needs to define variability of parameters. For the example described above last 14 days were chosen for setting the control scale which has Mean=181.0 and the fluctuation interval 2S=0.4 lb. So, the precision of the control scale gets higher in 2.5 times as compared to statistics for the whole month.

This method of setting the control scale does not provide high statistical validity as it is in QC but supports the priority of last results for weight monitoring.

To calculate statistics everyone can use computer programs or any usual calculator with statistical functions. Then the control scale is constructed for four zones above Mean such as +M, +1S, +2S and +3S and four zones below Mean such as −M, −1S, −2S and −3S using value of standard deviation to define control limits. The control limits for each zones are shown in the Table 2 which also includes limits calculated for an example with Mean=180 lb and S=0.4 lb. To make visual control for a weight plot easier a different color code for each control zone is proposed. The preferred variant of the color code is also shown in the Table 2.

TABLE 2
The limits and color code for the zones of the control scale

| | | | Zone limits for |
|---|---|---|---|
| | Zone limits | | control scale with |
| | (lower limit < W < upper limit) | | Mean = 180 lb |
| Zone Index | (W − body weight) | Color code | and S = 0.4 lb |
| +3S | W > Mean + 3S | Red (R) | W > 181.2 |
| +2S | Mean + 2S < W < Mean + 3S | Orange (O) | 180.8 < W < 181.2 |
| +1S | Mean + 1S < W < Mean + 2S | Yellow (Y) | 180.4 < W < 180.8 |
| +Mean | Mean < W < Mean + 1S | Light Green (LG) | 180.0 < W < 180.4 |
| −Mean | Mean − 1S < W < Mean | Green (G) | 179.6 < W < 180.0 |
| −1S | Mean − 2S < W < Mean − 1S | Blue (B) | 179.2 < W < 179.6 |
| −2S | Mean − 3S < W < Mean − 2S | Dark Blue (DB) | 178.8 < W < 179.2 |
| −3S | W < Mean − 3S | Violet (V) | W < 178.8 |

### 3. Setting up Control Rules for Weight Gain.

As per QC, there is no difference whether positive or negative deviations come in. For the weight control this switch is critical. Therefore the control rules were separated for positive and negative weight shifts.

The designation of rules is simplified as compared to Westgard rules. However, their formulas left the same. The main set of control rules for a weight gain comprises 6 rules. Every time when the weight violates any rule it means out-of-control state or a real shift up.

The description of control rules is shown in the Table 3. Their graphical presentations for the example with Mean 180 lb and S=0.4 lb are on FIG. 4.

TABLE 3
Control Rules for weight gain detection.

| Control | | Draw- |
|---|---|---|
| Rule | Description | ing |
| +4S | The weight jumps up on more than 4S units as | FIG. 4A |
| | compared to the previous value but doesn't exceed +3S | |
| | limit. It comprises two zone-to-zone transitions such as | |
| | −2S to +2S and −3S to +1S. | |
| +3S | The single weight value regardless of previous one | FIG. 4B |
| | exceeds +3S limit getting into +3S zone. | |
| +2S | Two consecutive results exceed +2S limit and fall into | FIG. 4C |
| | +2S zone. | |
| +1S | Four consecutive measurements exceed +1S limit and | FIG. 4D |
| | fall into +1S zone. | |
| +M | A row of six consecutive measurements come above | FIG. 4E |
| | Mean into +Mean zone. | |
| +T | Seven consecutive measurements get progressively | FIG. 4F |
| | higher and set a trend upward. | |

So, even smallest positive deviation repeated six times relates to the real weight shift as to the +M rule. It shows the detection limit for weight shifts eventually equals the sensitivity of the weight scale which now consists of 0.2 lb. This limit is more than 10 times lower as compared with detection limit 2.5 lb for previous art. The same way it improves feedback control for any diet and exercise program.

### 4. Setting up Control Rules for Weight Loss.

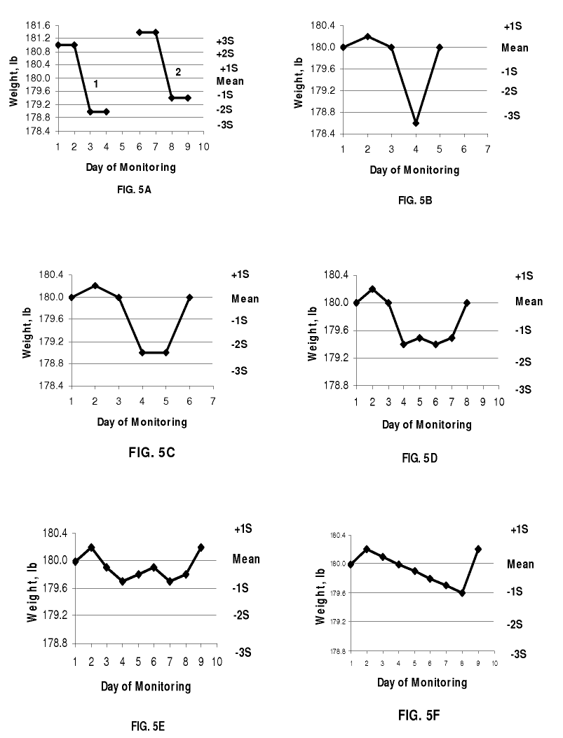

The similar set of the control rules for negative weight shifts is defined as shown in the Table 4 and on FIG. 5. The description for the example with Mean=80 lb and S=0.4 lb is also included. Every time when the weight violates any rule it means on-target state or a real shift down.

TABLE 4
Control Rules for weight loss detection.

| Control | | Draw- |
|---|---|---|
| Rule | Description | ing |
| −4S | The weight drops dawn on more than 4S units as | FIG. 5A |
| | compared to the previous value but falls above −3S | |
| | limit. | |
| | It comprises two zone-to-zone transitions: +2S to −2S | |
| | and +3S to −1S. | |
| −3S | The single weight value regardless of previous one | FIG. 5B |
| | falls below −3S limit into −3S zone. | |
| −2S | Two consecutive results drop below −2S limit and fall | FIG. 5C |
| | into −2S zone. | |
| −1S | Four consecutive measurements drop −1S limit and fall | FIG. 5D |
| | into −1S zone. | |
| −M | A row of six consecutive measurements come below | FIG. 5E |
| | Mean into −Mean zone. | |
| −T | Seven consecutive measurements get progressively | FIG. 5F |
| | lower and set a trend downward. | |

### 5. Logging on and Evaluating Results.

Once the control scale is set up and control rules are established the person can start logging results of measurement on a spreadsheet and plotting a graph. The separate forms are used for each monthly run.

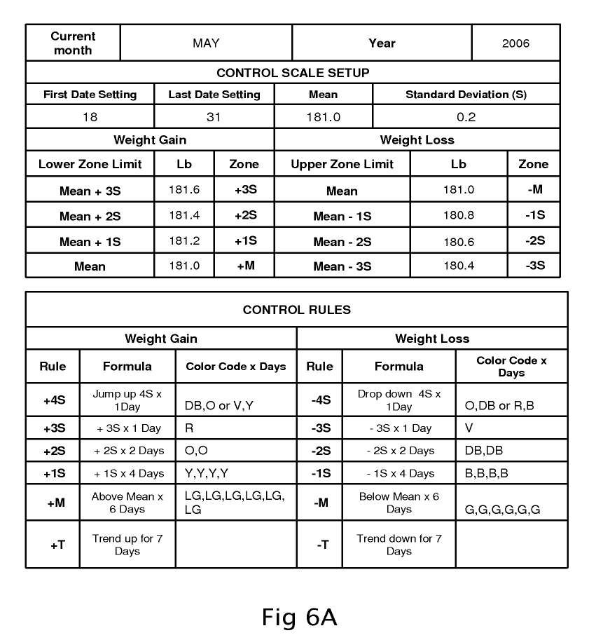

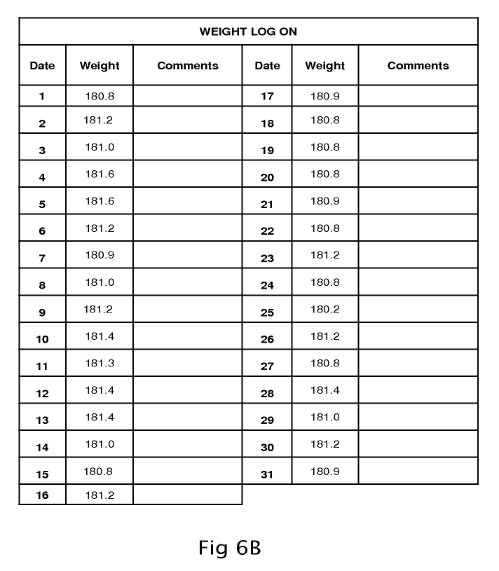

The preferred template form to log data on is shown on FIG. 6A and FIG. 6B which comprise following parts:

a/Identification of a run by the name of the current year and the current month to be monitored.
b/Control scale setup included:
Fist and last date of previous month used as a setting period for statistics calculation.
Mean of results for the setting period.
Standard deviation of results for the setting period.
Control limits and color code for +M, +1S, +2S, +3S zones related to the weight gain.
Control limits and color code for −M, −1S, −2S, −3S zones related to the weight loss.
   c/Control rules separated in turn on two groups for the weight gain and the weight loss.
   The weight gain group includes identification, a formula and color code presentation for following rules: +4S, +3S, +2S, +1S, +M and +T
   The weight loss group includes identification, the formula and color code presentation for following rules: −4S, −3S, −2S, −1S, −M and −T.
   d/Data log on presented by a table which includes but is not limited to separate columns for date, weight and comments. Each line relates to the date of month starting from 1 and ending with 31.

The person puts results of measurement into table and evaluates weight shifts by using control rules. The example of using the template is shown on the FIG. 6 for the control scale which is set up as to FIG. 3 for Mean=181.0 lb and S=0.2 lb.

By using color codes the weight shifts can be detected even without plotting a graph. The person marks a weight value with a color corresponding to the appropriate control zone and then checks the color code for control rules. For example, four yellow weight values (Y,Y,Y,Y) represent +1S weight gain then two dark blue weight values (DB, DB) represent −2S weight loss.

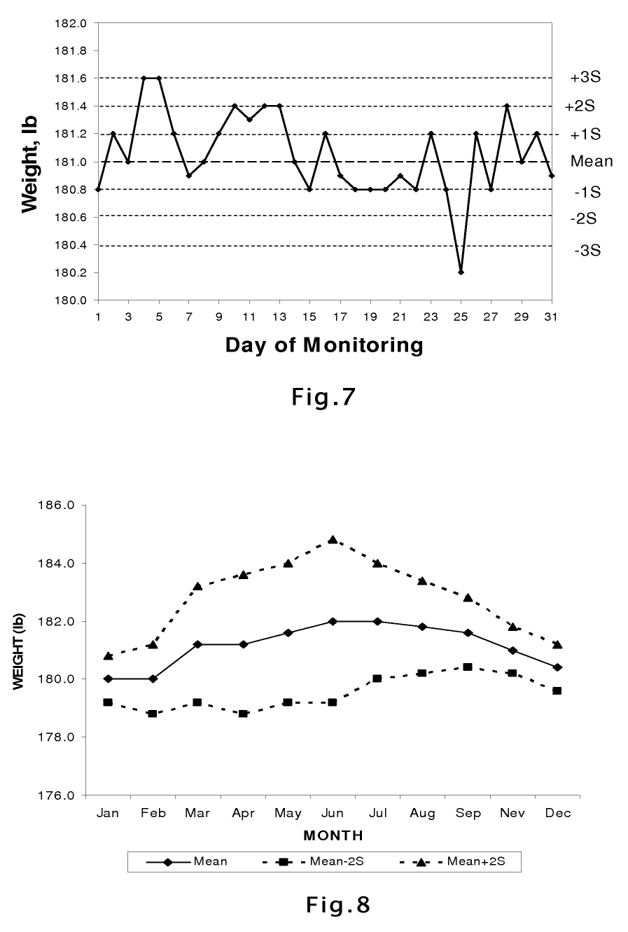

The preferred template graph to plot data on is shown on FIG. 7 and comprises following parts:

a/X-axis included all days of monitoring for current month starting from 1 and ending with 31.
b/First permanent Y-axis related to S-scale of variability. It includes following limits +4S, +3S, +2S, +1S, M, −1S, −2S, −3S and −4S as mandatory. The larger and smaller limits can be also included into graph. For better visual presentation of results the +3S, +2S, +1S, +M, −M, −1S, −2S, −3S control zones can be colored in different colors, for example as to color code described above.
c/Second Y-axis related to values of weight specified for each new control scale constructed. The person constructs his/her own control scale by putting calculated results for each control limits such as Mean, Mean−1S, Mean+1S and so on.

Then the person plots graph and evaluates weight shifts using control rules. For the example described the person identifies three out-of control states such as +2S shift on days 4-5, +1S shift on days 10-13 and +4S jump on day 26. The same way he or she identifies two on-target states such as −1M shift on days 17-22 and −3S jump on day 25.

When month is over, the person visually identifies a setting period for constructing a new control scale by choosing last results with no evident weight trend. Then the new scale is set up and a new cycle of weight monitoring starts up from the first date of the new month.

### 6. Evaluating Monthly Progress and Efficacy of the Weight Control.

The person can evaluate monthly progress by several ways:

a Mean and 2S value as a 95% confidence interval for random fluctuations for a setting period in the end of the month. It focuses on the final result of monthly run.
b/Mean and 2S value as the fluctuation interval for whole month. It gets overall estimation of weight load for the current month.
c/Calculating +Score as a sum of positive shifts, −Score as the sum of negative shifts and Total Score as the sum of +Score and −Score over a run.

The Mean value gets statistically verified estimation of variable weight and excludes any random error typical for single measurements. The weight variability determined by the 2S fluctuation interval is the second important controlled parameter. The high variability contributes into high chances the weight slips out of control. Therefore the first step in taking weight under control should include decreasing weight variability.

The template form and chart for tracking monthly results are shown in the Table 5 and on FIG. 8. The person should indicate in the form if there is statistics for a whole run or for the last interval. The template form comprises separate columns for the name of month, Mean, S, Mean +2S, Mean −2S and comments. The template chart comprises X-axis for at least 12 months and Y-axis for the weight. The person plots three curves for Mean, Mean +2S and Mean −2S. Then he or she can visually evaluate changing both a mean weight value and its variability.

TABLE 5
Template form for tracking monthly results

| Month | Mean | S | Mean − 2S | Mean + 2S | Comments |
|---|---|---|---|---|---|
| January | 180.0 | 0.4 | 179.2 | 180.8 | |
| February | 180.0 | 0.6 | 178.8 | 181.2 | |
| March | 181.2 | 1 | 179.2 | 183.2 | |
| April | 181.2 | 1.2 | 178.8 | 183.6 | |
| May | 181.6 | 1.2 | 179.2 | 184 | |
| June | 182.0 | 1.4 | 179.2 | 184.8 | |
| July | 182.0 | 1 | 180 | 184 | |
| August | 181.8 | 0.8 | 180.2 | 183.4 | |
| September | 181.6 | 0.6 | 180.4 | 182.8 | |
| November | 181.0 | 0.4 | 180.2 | 181.8 | |
| December | 180.4 | 0.4 | 179.6 | 181.2 | |

The example presented in Table 5 and FIG. 8 shows benefits of using both Mean and variability parameters: the rising variability in that case is one of the reasons of the weight gain and on the contrary lowering variability can be considered as a possible cause for the weight loss.

Calculating scores of weight shifts provides overall estimation of all failures and successes over the run. Each real shift is represented by the score such as a value specified for different shifts multiplied by days of that shift. This equation takes into account the possibility for the shift duration exceeded minimal interval required. A Score Scale is shown in the Table 6.

TABLE 6
Score scale for different weight shifts.

| | Control Zone | | | | | | | | | |
|---|---|---|---|---|---|---|---|---|---|---|
| | +4S | +3S | +2S | +1S | +M | −M | −1S | −2s | −3S | −4S |
| Score | +4 | +3 | +2 | +1 | +0.5 | −0.5 | −1 | −2 | −3 | −4 |

Positive Total Score indicates prevalence of weight gains over weight losses. Otherwise, negative Total Score indicates prevalence of weight losses over weight gains.

Accordingly, for the example from FIG. 6B and FIG. 7 these scores are:

### +Score=+2*2+1*4+4*1=+12

which summarizes +2S, +1S and +4S shifts;
−Score=−0.5*6−3*1=−6
which summarizes −1M and −3S scores;
Total Score=(+Score)+(−Score)=12−6=6
which indicates prevalence of weight gains over weight losses.

### Conclusion, Ramifications, and Scope

The present invention proposes a new approach to the control of the weight and weight-dependent parameters based on a conception of Multirule Quality control. It is not just transfer of this conception to a new subject. Due to controlling variability of an object instead of a procedure new strong requirements for the length of setting and monitoring periods are applied with a priority of last results of measurements instead of statistical validity. Then two groups of control rules specified for weight gain and weight loss and three control zones such as in-control, out-of-control and on-target are set up.

As a result a personalized control scale to detect small shifts of weight parameters among their random fluctuations is constructed, which is important for promptly correction actions to be applied. The new approach to the weight control essentially changes assessment of its progress focusing on the final result of monthly run and the correction of unwanted deviations within the run. The variability of results, +Score, −Score and Total Score for weight shifts as well as new parameters such as Fat Mass and FMI become new important characteristics to be controlled. Special template forms to log on and evaluate data are developed which comprise sections for setting a control scale, designation of control rules for weight gain and weigh loss, color code for control zones, and logging on or plotting results. They make detection of real weight shifts easy and visual.

The essential advantage of present invention is wide possibilities for its embodiment which comprises but is not limited to a) paper template forms separated or inserted to any printed materials; b) algorithms for computer programs inserted to a weight scale and other measuring devises, personal computer, PDA and other electronic devises, and c) algorithms for programs provided direct data transfer from measuring devices to a computer.

While above description contains many specificities, these should not be construed as limitations on the scope of the invention. Many other variations are possible. For example, the +M and −M rules can imply 8 or 10 consecutive measurements above or below mean as to Westgard rules. The cumulative statistics comprising data for current run can be applied which obviously requires a special computer program to be used. The template forms can comprise different color code scheme for control zones as well as additional information such as diet, calorie intake, exercising and so on. The body weight can be measure in kg or other units.

Accordingly, the scope of the invention should be determined not by the embodiment illustrated, but by the appended claims and their legal equivalents.

What is claimed is:

1. A method for controlling a body weight or any weight-dependent parameters comprising the steps of:
a) consistent daily measuring said parameters;
b) setting monthly runs for monitoring said parameters;
c) constructing a new control scale for each month by using statistics such as mean and standard deviation for the last results of previous month with no evident trend and shift but no less than for 7 measurements;
d) identifying in-control, out-of-control and on-target states as to nonrandom shifts of the weight or weight-dependent parameters over the run by using control rules developed before for Quality control;
e) evaluating monthly progress in the weight control by calculating mean and variability of said parameters for the whole run or for the last interval with no evident trend and shift;
f) evaluating efficacy of the weight control by calculating a Score of weight shifts over the run.

2. The method of claim 1 wherein step (a) comprises daily measurement of said parameters for fasting body in the morning.

3. The method of claim 1 wherein step (a) comprises but is not limited to daily measurement of BMI, % Fat, % Body Water, Muscle Mass, Bone Mass, Visceral Fat as well as new parameters Fat Mass (FM) determined as % Fat multiplied by body weight and Fat Mass Index (FMI) determined as Fat Mass divided by square of height.

4. The method of claim 1 wherein step (c) comprises designation of control zones such as +3S, +2S, +1S, +M, −M, −1S, −2S and −3S with different color code for each zone.

5. The method of claim 1 wherein step (d) comprises designation of control rules for positive shifts of the weight and weight dependent parameters such as +4S, +3S, +2S, +1S, +M and +T and for negative shifts of said parameters such as −4S, −3S, −2S, −1S, −M and −T.

6. The method of claim 1 wherein step (d) comprises a template form for monthly run to log data on included identification of a run, control scale setup, a formula and a color code for each control rule and a table to put daily results.

7. The method of claim 1 wherein step (d) comprises a template graph for monthly run to plot data on included:
a/X-axis for days of monitoring;
b/first Y-axis with permanent S-scale and control limits for +3S, +2S, +1S, M, −1S, −2S and −3S where each control zone is colored with different color;
c/second Y-axis related to values of the weight parameters specified for each new scale constructed by indicating said values for each control limit of S-scale.

8. The method of claim 1 wherein step (f) comprises calculating +Score as a sum of positive weight shifts, −Score as the sum of negative weight shift and Total Score as the sum of +Score and −Score over the run where the score for each shift is the value specified for the different shifts multiplied by days of the shift.

9. The method of claim 1 comprises but is not limited to embodiments such as:
a/paper template forms implemented separately or inserted into books or other printed materials;
b/algorithms for computer programs inserted into the weight scale and other measuring devices, personal computer, PDA and other electronic devices;
c/algorithms provided direct data transfer from measuring devices to the computer,
whereby a person can early identify small real shifts of weight parameters among their random fluctuations by using personalized control scale based on own variability of said parameters and special control rules, and can promptly correct effector elements of weight control such as eating and exercising.
